(19) United States
(12) Patent Application Publication
(10) Pub. No.: US 2012/0215212 A1
(43) Pub. Date: Aug. 23, 2012

(54) TREATMENT FOR PULMONARY DISORDERS

(76) Inventors: Michael M. Selzer, Wayzata, MN (US); Mark C. Johnson, Phoenix, AZ (US); Erik N.K. Cressman, Lake Elmo, MN (US)

(21) Appl. No.: 13/299,135

(22) Filed: Nov. 17, 2011

Related U.S. Application Data
(60) Provisional application No. 61/414,731, filed on Nov. 17, 2010.

(51) Int. Cl. A61B 18/04

(52) U.S. Cl. 606/27

(57) ABSTRACT

A thermochemical ablation system can be used to ablate a portion of a bodily structure, such as a human airway. In some examples, the thermochemical ablation system includes a first ablation reagent, a second ablation reagent, and an expandable balloon positioned adjacent the distal end of a catheter. The expandable balloon can be inserted into the bodily structure and the two ablation reagents combined to cause an exothermic reaction that generates heat. The heat may create a substantially uniform temperature distribution across the surface of the expandable balloon, providing substantially uniform ablation of tissue adjacent the balloon.

This application claims the benefit of U.S. Provisional Application No. 61/414,731, filed Nov. 17, 2010, the entire content of which is incorporated herein by reference.

### TECHNICAL FIELD

This disclosure relates to medical devices and methods, more particularly, to medical devices and methods for treating pulmonary disorders.

### BACKGROUND

It is estimated that there are over 300 million asthma patients worldwide with over 20 million in the United States alone. Experts indicate that over the past decade the prevalence of asthma has increased 50 percent. The strongest risk factors for asthma appear to be a combination of genetic predisposition with environmental exposure to inhaled substances and particles. Asthma is typically characterized by broncho-constriction, excessive mucus production, inflammation and swelling of airways. These conditions can cause widespread and variable airflow obstruction, making it difficult for the asthma sufferer to breathe. It is reported that asthma management consumes more than $18 billion of health care resources each year in the U.S. Patients with severe persistent asthma exhibit a lack of asthma symptom control over both short periods (2-3 months) and extended timeframes. In persistent severe patients, there is a general increase in the bulk (hypertrophy and hyperplasia) of the airway smooth muscle in the large bronchi. Also, the smaller airways of these patients are typically narrowed and show inflammatory changes. In the United States and Europe, this stage of asthma represents over 6 million patients, 5,500 asthma deaths annually, and approximately 800,000 annual adult hospitalizations.

### SUMMARY

In general, the disclosure describes techniques for thermochemically ablating tissue defining a wall of a lumen of a mammalian body such as, e.g., a human airway. In some examples of the described techniques, an expandable balloon connected to a catheter is inserted into an airway of a patient. Two or more ablation reagents are combined to cause an exothermic reaction that generates heat. The ablation reagents may be combined in the expandable balloon or introduced into the balloon after combination so as to heat an exterior surface of the balloon. Regardless, the balloon can be expanded, e.g., upon introducing the ablation reagents under pressure into the balloon, so the balloon conforms to a size and shape of the lumen (e.g., airway) into which the balloon is inserted.

Thermal energy applied to the airway wall of the patient through the expandable balloon may heat endothelium and smooth muscle layers of the airway to a temperature sufficient to result in their eventual obliteration. However, the temperature may be low enough that surrounding tissues beyond the adventitia and parenchyma are not heated to levels that can injure the tissues. For example, an exterior surface of the expandable balloon may be heated to a temperature ranging from approximately 45 degrees Celsius to approximately 75 degrees Celsius, and this temperature may be applied to the airway wall for a period ranging from approximately 5 seconds to approximately 25 seconds. These or other temperatures may shrink or destroy tissue responsible for contracting (e.g., smooth muscle) during an asthmatic attack, minimizing or eliminating airway constriction attendant to muscle contraction during an asthmatic attack.

In one example, the disclosure describes an ablation system that includes a first reservoir configured to house a first ablation reagent, a second reservoir configured to house a second ablation reagent, and a catheter. According to the example, the second ablation reagent is configured to generate an exothermic reaction when mixed with the first ablation reagent. The example further specifies that the catheter is configured to be inserted into at least a portion of a human airway and that the catheter extends from a proximal end to a distal end and includes an expandable balloon adjacent the distal end. The catheter is configured to provide fluid communication between the first reservoir, the second reservoir, and an interior of the expandable balloon.

In another example, a method is described that includes delivering a first ablation reagent into a catheter inserted into at least a portion of a human airway and delivering a second ablation reagent into the catheter. According to the example, the catheter extends from a proximal end to a distal end and includes an expandable balloon adjacent the distal end. The example further specifies that the second ablation reagent is configured to generate an exothermic reaction when mixed with the first ablation reagent.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram of an example ablation system including an ablation reagent delivery device that may be used to thermochemically ablate tissue in the airway or other bodily lumen of a patient.

FIG. 1B is a perspective view of an example ablation reagent delivery device.

FIG. 2 is a plot of example time, temperature, and concentration date for exothermic reactions that may be generated during thermochemical ablation using the ablation system of FIG. 1.

FIG. 3 is a functional block diagram illustrating an example of the ablation reagent delivery device of FIG. 1.

FIGS. 4A and 4B are corresponding cross-sectional diagrams of an example expandable balloon that may be used in the ablation system of FIG. 1

FIG. 4C is a sectional view of an expandable balloon and catheter that may be used to thermochemically ablate tissue in the airway or other bodily lumen of a patient.

FIG. 5 is a plot of an example relationship between a size of an exhaust lumen and an expandable balloon length for an example ablation system.

FIG. 6 is a flow chart of an example method for thermochemically ablating tissue.

FIG. 7 is a cross-sectional view of an expandable balloon in an airway.

### DETAILED DESCRIPTION

Devices, systems, and techniques for thermochemically ablating a portion of a bodily structure, such as a human airway, are described. As described in some examples herein, a thermochemical ablation system may include a first reservoir that houses a first ablation reagent and a second reservoir that houses a second reagent. The first and second reservoirs can be fluidly connected to a catheter that includes an expandable, fluid-impermeable structure, such as a balloon, positioned adjacent a distal end of the catheter. In operation, the catheter can be inserted into the bodily structure, such as a portion of a bronchial tree, and expanded by injecting the first and second ablation reagents into the balloon. Upon mixing, the first and second ablation reagents can exothermically react, releasing heat which migrates through the balloon wall and causes an exterior surface of the balloon to increase in temperature to a temperature sufficient to ablate tissue surrounding the balloon.

The devices, systems, and techniques described herein can be used to treat a variety of different disorders including pulmonary disorders such as, e.g., asthma. Asthma is typically characterized as an inflammation of the airway that causes resistance to airflow within the lungs. In some patients, chronic and persistent asthma can lead to structural changes of the airway wall, further affecting the function of the airway wall and the influence of airway hyper-responsiveness. For example, smooth muscle lining an airway wall can increase in bulk, e.g., due to hypertrophy and/or hyperplasia, in an asthmatic individual, further exacerbating the asthma condition. Ablation of this tissue can reduce or eliminate the number of asthmatic episodes experienced by the asthmatic individual, e.g., by destroying the smooth muscle that constricts the airway walls.

In accordance with the techniques described in some examples of the present disclosure, a catheter that includes an expandable balloon in fluid communication with two or more ablation reagents can be inserted into the airway of an asthmatic patient. A bronchoscope or other delivery device may or may not be first inserted into the patient to help guide insertion of the catheter. In either case, the expandable balloon can be expanded once positioned at a location in the airway of the patient to be ablated. In some examples, the first and/or second ablation reagents can be introduced into the expandable balloon, for example under pressure, to expand the balloon. The expandable balloon may expand until it conforms to and is in contact with the wall of the airway along its working length. Further, the first and second ablation reagents, which may be mixed in the expandable balloon itself or outside of the balloon, may cause an exothermic reaction, heating the exterior surface of the expandable balloon to a temperature sufficient to ablate tissue adjacent the expandable balloon.

Ablating tissue in the airway of an asthmatic patient may heat endothelium and smooth muscle layers of the airway to a temperature sufficient to destroy at least a portion of the endothelium and smooth muscle tissues. These tissues may be contract and constrict the airway, e.g., during an asthmatic attack. Accordingly, destroying the tissues can minimize or eliminate airway constriction that may be associated with asthmatic symptoms and/or asthmatic attack. In some embodiments, physiological responses to the ablating heat may also include crosslinking sub-mucosa and/or collagen present in the anatomical structure of the airway to stiffen the airway.

A thermochemical ablation system according to some examples of the present disclosure may generate a substantially uniform temperature (e.g., temperature profile) across a body of a heating member. For example, an exterior surface of an expandable balloon of the thermochemical ablation system may heat to a temperature that is substantially constant across the surface of the balloon, both circumferentially and longitudinally. In some examples, no portion of the exterior surface of the expandable balloon heats to a temperature above a threshold value. Such a uniform temperature distribution across the surface of the expandable balloon may provide a substantially uniform ablation of tissue adjacent the balloon in the airway of the patient. In contrast to other ablation systems such as radio frequency (RF) ablation systems, which may have hot spots or other temperature discontinuities that cause ablation treatment gaps across both the circumference and length of the region of the airway being treated, a system in accordance with examples of the disclosure may substantially uniformly ablate tissue in the region being heated. This may lead to a more efficacious therapeutic result for the patient undergoing treatment.

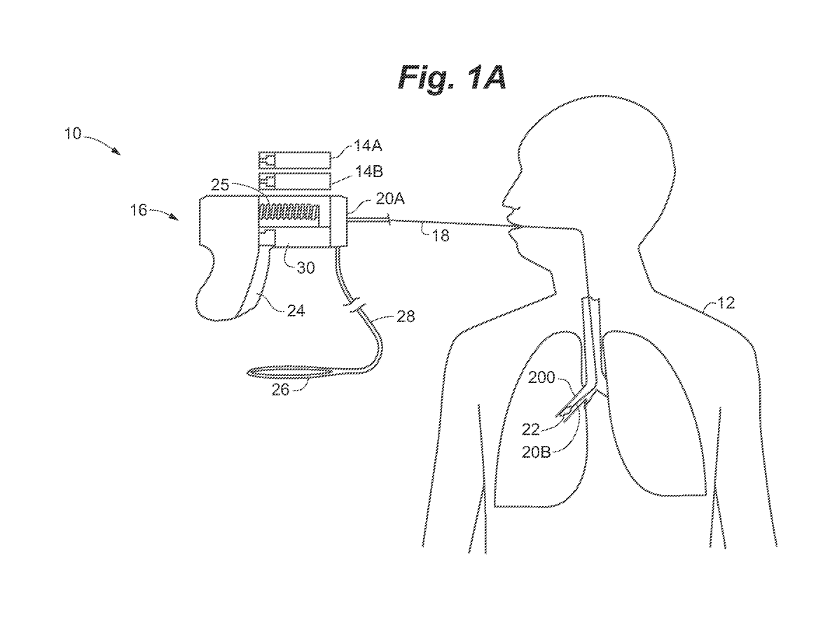

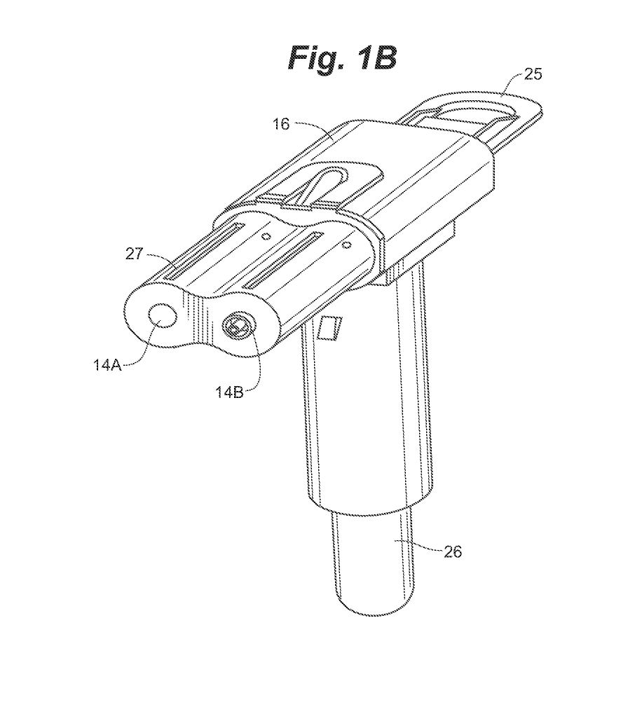

FIG. 1A is a conceptual diagram of an example ablation system 10 that may be used to thermochemically ablate tissue in the airway or other bodily lumen of patient 12. Ablation system 10 includes at least two ablation reagents which, in the example of FIG. 1A, are illustrated as first and second ablation reagents 14A and 14B (collectively “ablation reagents 14”). Ablation system 10 also includes an ablation reagent delivery device 16 (hereinafter “delivery device 16”) that is that is connected to at least one catheter 18. Catheter 18 extends from a proximal end 20A connected to delivery device 16 to a distal end 20B positioned at or adjacent a target ablation site within patient 12. Catheter 18 is configured to deliver ablation reagents 14 from outside of patient 12 to the target ablation site within the patient. In the example shown in FIG. 1A, the target ablation site is within a portion of the bronchial tree of patient 12. In other examples, the target ablation site may be other sites within the airway of patient 12 or a different lumen of the body of patient 12. In addition, ablation reagents 14 are illustrated within reservoirs that separate from and insertable into delivery device 16. In other examples, reservoirs housing ablation reagents 14 may be permanently affixed to delivery device 16 or other configurations, as described below.

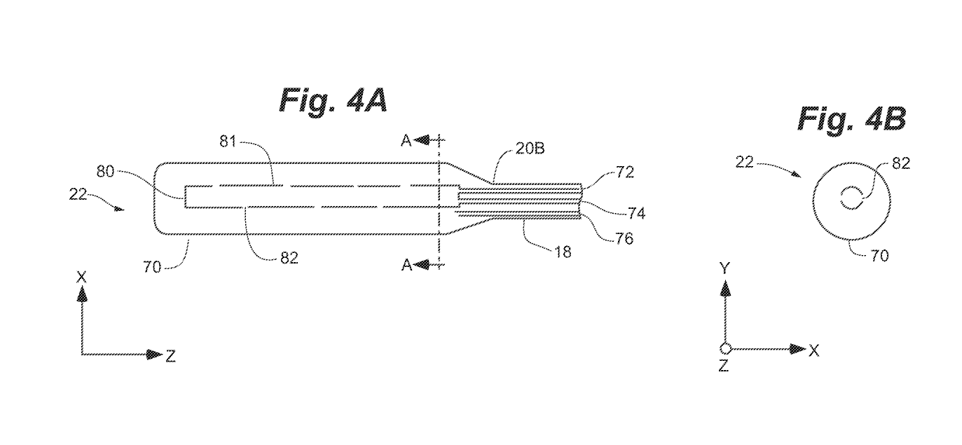

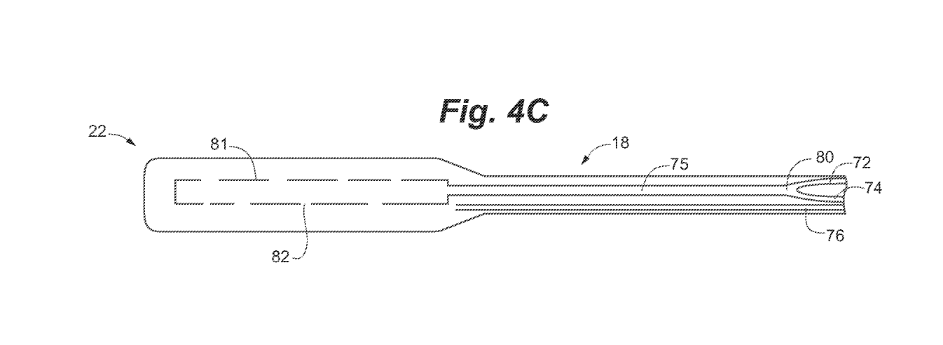

As described in greater detail below with respect to FIGS. 4A and 4B, catheter 18 includes an expandable balloon 22 positioned adjacent the distal end 20B of the catheter. Expandable balloon 22 may define a fluid-impermeable structure that is configured to receive ablation reagents 14 and expand in response to the pressure of the reagents. In some examples, expandable balloon 22 is configured to expand so as to conform to the portion of the bronchial tree into which the catheter is inserted. For example, expandable balloon 22 may expand until an exterior surface of the balloon contacts a wall of a bronchial tree lumen about substantially an entire perimeter of the balloon, e.g., closing fluid movement through the bronchial tree lumen. Regardless, expandable balloon 22 may transfer thermal energy generated by reaction of ablation reagents 14 to the target ablation site within patient 12. This thermal energy may heat tissue in the patient at the target ablation site so as to ablate the tissue.

Delivery device 16 delivers first ablation reagent 14A from a first reservoir and second ablation reagent 14B from a second reservoir to patient 12 through catheter 18. Catheter 18 can comprise a unitary catheter or a plurality of catheter segments connected to form an overall catheter length. In addition, catheter 18 may be a single-lumen catheter or a multi-lumen catheter. With a single lumen catheter, delivery device 16 may be configured to mix first ablation reagent 14A with second ablation reagent 14B in or adjacent proximal end 20A of catheter 18 and deliver the mixture of ablation reagents (or reaction product thereof) to expandable balloon 22. When configured with a multi-lumen catheter, catheter 18 may include a lumen fluidly connecting a reservoir housing first ablation reagent 14A to expandable balloon 22 or a separate location in the body of patient 12 (e.g., a mixing zone located proximally of expandable balloon 22). In this example, catheter 18 may also include a separate lumen fluidly connecting a reservoir housing second ablation reagent 14B to expandable balloon 22 or a separate location in the body of patient 12 (e.g., a mixing zone located proximally of expandable balloon 22). Catheter 18 may include additional lumens, e.g., connected to one or more additional ablation reagents, for removing fluids from expandable balloon 22 so as to deflate the balloon, and/or receiving a guide wire, or the like.

Delivery device 16 can be configured to manually deliver ablation reagents 14 into the body of patient 12 or to automatically delivery the ablation reagents into the body, either in batch or continuously to maintain a desired pressure in the expandable balloon and/or heat profile. In example of FIG. 1A, delivery device 16 is illustrated as a manual delivery device that includes an acuatable trigger 24 and a plunger 25. Actuatable trigger 24 is coupled (e.g., mechanically and/or electrically) to a reservoir housing (e.g., a cartridge) first ablation reagent 14A and a reservoir housing (e.g., a cartridge) second ablation reagent 14B. In operation, a user may apply a force to actuatable trigger 24 so as to cause first ablation reagent 14A and/or second ablation reagent 14B to be delivered under pressure into catheter 18. For example, a user may apply a force to actuatable trigger 24 so as to cause plunger 25 to advance and discharge first ablation reagent 14A and/or second ablation reagent 14B under pressure into catheter 18. In some examples, actuation of actuatable trigger 24 causes first ablation reagent 14A and second ablation reagent 14B to be simultaneously delivered into catheter 18. In other examples, first ablation reagent 14A and second ablation reagent 14B may be independently and sequentially delivered into catheter 18. For example, actuatable trigger 24 may comprise two or more triggers and two or more plungers, such as one trigger and one plunger coupled to or operatively associated with a reservoir housing first ablation reagent 14A and another trigger and another plunger coupled to or operatively associated with a reservoir housing second ablation reagent 14B. In such an example, a user may independently actuate each trigger, e.g., so as to independently control the timing and/or rate at which first ablation reagent 14A and second ablation reagent 14B are delivered into catheter 18. While actuatable trigger 24 is shown in the style of a trigger, in other examples, other manual actuation devices such as, e.g., buttons, knob, or lever may be used in additional to or in lieu of a trigger, and it should be appreciated that the disclosure is not limited in this respect.

Another embodiment of a delivery device 16 is shown in FIG. 1B. In the example of FIG. 1B, delivery device 16 includes a break-action mechanism to access the reservoir housing first ablation reagent 14A and the reservoir housing second ablation reagent 14B. In operation, a user pulls a lever 25 back to release pressure from springs (not shown) pushing against the reservoirs. The user then activates the break-action mechanism to tilt a reservoir housing portion 27 of the delivery device with respect the remainder of the delivery device. The user may then refill the fluid reservoirs (e.g., by inserting new cartridges), and close the break-action mechanism.

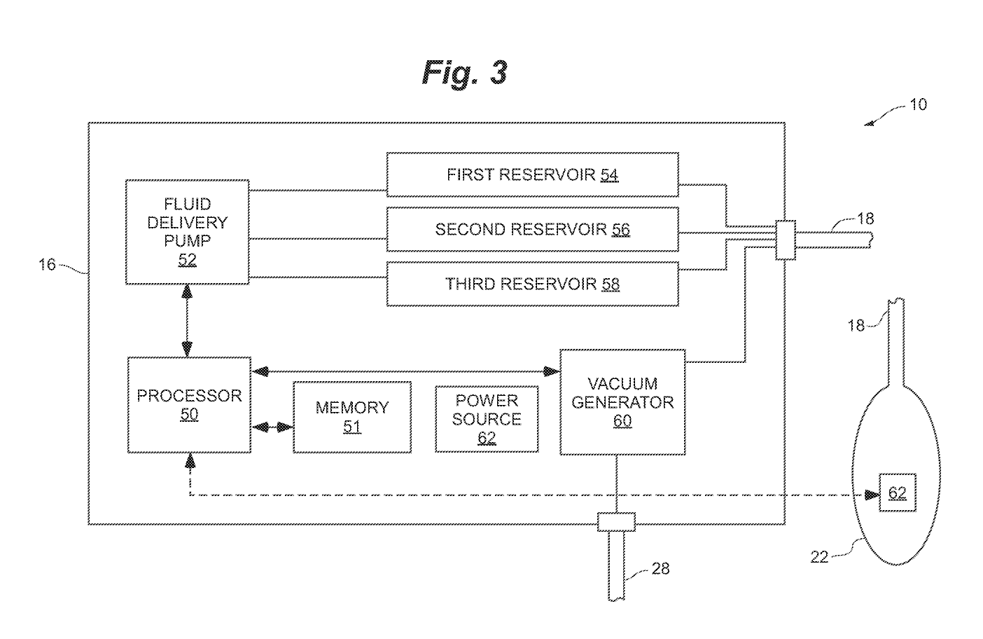

Further, although delivery device 16 is illustrated in the style of a delivery gun that is manually operable to deliver ablation reagents to expandable balloon 22, other manual and non-manual delivery devices are both possible and contemplated. For example, delivery device 16 may be a syringe with plunger that can be depressed to dispense ablation reagents 14 into catheter 18. In another example, ablation reagents 14 may be housed under pressure and delivery device 16 may be a device configured to open a conduit (e.g., open a valve, puncture a seal) so as to deliver the ablation reagents into catheter 18. In still other examples, delivery device 16 may include a computer-controlled mechanism that acts, in response to a user input, to deliver first ablation reagent 14A and/or second ablation reagent 14B under pressure into catheter 18 (see FIG. 3).

Delivery device 16 may include reservoirs for storing first ablation reagent 14A and second ablation reagent 14B. Further, delivery device 16 may include more than two reservoirs (e.g., three, four, five or more reservoirs) for storing more than two types of ablation reagents or for storing different amounts of ablation reagents. Ablation reagents 14 may be stored in replaceable or non-replaceable reservoirs. Example replaceable reservoirs include bags, cartridges, and syringes. In some examples, ablation reagents 14 are stored in reservoirs that are configured to contain a pre-measured or preheated (e.g., to 37 degrees C., etc.) amount of ablation reagent.

Ablation system 10 in the example of FIG. 1A is configured to thermally ablate tissue in patient 12 by chemically reacting one or more reagents (e.g., first ablation reagent 14A) with one or more other reagents (e.g., second ablation reagent 14B). Accordingly, the heat generated by the reaction of these reagents may be sufficient to cause an exterior surface of expandable balloon 22 (e.g., a surface in contact with an airway wall of patient 12) to increase in temperature to a temperature sufficient to ablate tissue of the bodily structure adjacent to and/or in contact with the expandable balloon. In some examples, first ablation reagent 14A is an acid while second ablation reagent 14B is a base. The strength and chemical composition of the acid and base may vary depending, e.g., on the desired ablation temperature.

Example acids for first ablation reagent 14A may include (or, in other examples, be selected from the group consisting of) acetic acid, peracetic acid, hydrochloric acid, hydrobromic acid, hydriodic acid, sulfuric acid, nitric acid, nitrous acid, perchloric acid, phosphoric acid, oxalic acid, pyruvic acid, malonic acid, and amino acids (e.g., carboxylic acid derivatives). Example bases for second ablation reagent 14B may include (or, in other examples, be selected from the group consisting of) KOH, NaOH, NH₄OH, Ca(OH)₂, NaHCO₃, K₂CO₃, BuLi, NaOEt or NaSEt (e.g., Na or K salts of alkoxides or thio analogues), NaH, KH, and amines. In one example, first ablation reagent 14A is selected from the group consisting of HCl, AcOH (acetic hydroxide), and citric acid. In this example, second ablation reagent 14B may include or be an alkali metal hydroxide (e.g., NaOH, KOH). Additional ablation reagents that can react to thermochemically ablate tissue and that may be used in ablation system 10 are described in US Patent Publication Nos. 2010/145304 and 2011/152852, and PCT Publication WO 2011/066278. The entire contents of these publications are incorporated herein by reference.

Although the concentration and chemical composition of first ablation reagent 14A and second ablation reagent 14B can vary, e.g., based on the desired temperature to which the exterior surface of expandable balloon 22 is to be elevated, in some examples, first ablation reagent 14A and second ablation reagent 14B are selected to cause an exothermic reaction (i.e., upon mixing) that heats the exterior surface of expandable balloon 22 to a temperature greater than 45 degrees Celsius. For example, first ablation reagent 14A and second ablation reagent 14B may be selected to cause an exothermic reaction that heats the exterior surface of expandable balloon 22 to a temperature greater than 55 degrees Celsius, greater than 75 degrees Celsius, or even greater than 100 degrees Celsius. In some additional examples, first ablation reagent 14A and second ablation reagent 14B are selected to cause an exothermic reaction that heats the exterior surface of expandable balloon 22 to a temperature ranging from approximately 45 degrees Celsius to approximately 110 degrees Celsius such as, e.g., a temperature ranging from approximately 55 degrees Celsius to approximately 65 degrees Celsius. The foregoing temperatures, when applied to tissue for an appropriate amount of time, may ablate endothelium and/or smooth muscle tissue within the wall of the airway of patient 12 without ablating tissues beyond the adventitia and parenchyma.

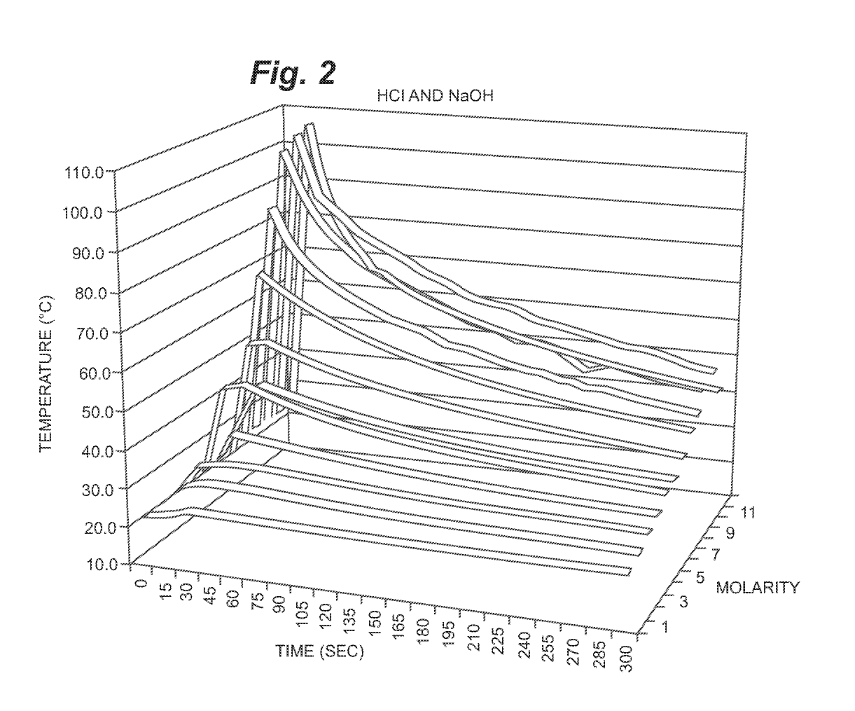

FIG. 2 is a plot of example time, temperature, and concentration date for exothermic reactions that may be generated during thermochemical ablation using ablation system 10. In particular, FIG. 2 illustrates example relationships between temperature and time for mixtures of different molarities of HCl and NaOH, where equal amounts of the HCl and NaOH having the same concentration are mixed together. FIG. 2 illustrates that temperatures greater than 100 degrees Celsius are achievable using common acids and bases for the exothermic reaction. FIG. 2 also illustrates that the exothermic reaction may have a peak temperature that occurs a given period after mixing two or more reagents and a steadily decreasing temperature after achieving the peak temperature.

In some examples, ablation reagents 14 may be selected to cause an exterior surface of the expandable balloon to increase to a temperature ranging from approximately 45 degrees Celsius to approximately 110 degrees Celsius for a period ranging from approximately 5 seconds to approximately 25 seconds. For example, ablation reagents 14 may be selected to cause an exterior surface of the expandable balloon to increase to a temperature ranging from approximately 5 degrees Celsius to approximately 65 degrees Celsius for a period ranging from approximately 8 seconds to approximately 12 seconds. Other temperature and time combinations are contemplated, however, and it should be appreciated that the disclosure is not limited in this respect.

With further reference to FIG. 1A, the heat generated from the chemical reaction of first ablation reagent 14A and second ablation reagent 14B may heat expandable balloon 22 in such a way that the temperature across a surface of the expandable balloon (e.g., an exterior surface of the balloon in contact with an airway wall) is substantially constant both about its circumference and along its length. For example, the heat generated by the chemical reaction may heat the expandable balloon so that no portion of the balloon is hotter than any other portion of the balloon by a threshold value. The threshold value may less then 20 degrees Celsius such as, e.g., less than 10 degrees Celsius, or less than 5 degrees Celsius, although other threshold values are also possible. Because expandable balloon 22 can have a number of different sizes and shapes, in some examples, the balloon is configured so that no surface of the balloon in contact with a portion of patient 12 (e.g., an airway wall of patient 12) is hotter than any other portion of the balloon in contact with a portion of patient 12 by the threshold value.

In some embodiments, the temperature of the expandable balloon can be altered or maintained by the continuous or substantially continuous introduction of ablation reagents or their reaction product into the expandable balloon during a procedure. In general, the greater the flow rate of a given ablation reagent set or their reaction product, the less volume of fluid will be required to maintain a desired expandable balloon temperature.

The substantially uniform temperatures that can be generated across expandable balloon 22 during chemical reaction may be useful to provide a substantially uniform ablation of tissue adjacent the balloon in the airway of patient 12. In contrast to other ablation systems such as radio frequency (RF) ablation systems, which may have hot spots or other temperature discontinuities that cause ablation treatment gaps across both the circumference and length of the region of the airway being treated, thermochemical ablation in accordance with examples of the disclosure may substantially uniformly ablate tissue in the region being heated. This may lead to a more efficacious therapeutic result for the patient undergoing treatment.

After thermochemically ablating tissue within patient 12, the reaction product of first ablation reaction 14A and second ablation reagent 14B may be exhausted (e.g., evacuated or removed) from expandable balloon 22 to at least partially, and in some cases fully, deflate the balloon. For this reason, delivery device 16 and/or catheter 18 can be configured to remove fluids from within expandable balloon 22 and to deliver the fluids outside of the body of patient 12. In some examples, the fluids within expandable balloon 22 are exhausted through the same lumen or lumens of catheter 18 used to deliver first ablation reagent 14A and second ablation reagent 14B to the balloon. The fluids from expandable balloon 22 may be returned to the reservoirs originally housing the ablation reagents, e.g., for disposal. Alternatively, delivery device 16 may include a diverter valve or other mechanism to direct the fluids from expandable balloon 22 to a drain port or waste storage structure.

In other examples, catheter 18 includes an exhaust lumen separate from the lumen(s) used to deliver ablation reagents 14 to the expandable balloon. The exhaust lumen can extend from proximal end 20A of catheter 18 to distal end 20B of the catheter, providing a fluid connection between an interior of expandable balloon 22 to outside of the body of patient 12. The exhaust lumen can be fluidly connected to a drain port or waste storage structure positioned outside of patient 12, e.g., to allow a user remove and dispose of fluids within expandable balloon 22.

In the example of FIG. 1A, ablation system 10 includes waste storage structure 26 connected to delivery device 16 by a conduit 28. Waste storage structure 26 may be a flexible bag, a rigid container, or other structure that is fluidly connected to catheter 18 via conduit 28. For example, waste storage structure 26 may be fluidly connected to an exhaust lumen extending through catheter 18 via conduit 28, or waste storage structure 26 may be fluidly connected to a lumen of catheter 18 used to deliver one or more ablation reagents 14 to expandable balloon. While waste storage structure 26 is shown as separate from delivery device 16 and connected by conduit 28, in other examples, waste storage structure 26 may be attached (e.g., permanently or removably) or integrated with delivery device 16 without requiring conduit 28, as shown in the embodiment of FIG. 1B.

Any suitable driving forces may be used to exhaust waste fluids from expandable balloon 22 and to deliver the waste fluids to waste storage structure 26. In some examples, waste fluids are passively removed from expandable balloon, e.g., without the application of a driving force from outside the body of patient 12. Under the influence of gravity and/or a pressure caused by an elasticity of the walls of expandable balloon 22, waste fluid within expandable balloon 22 may passively transport from expandable balloon 22 to waste storage structure 26.

In addition to or in lieu of passive driving forces, active driving forces may be used to exhaust waste fluid from expandable balloon 22. Active forces may be forces applied from outside of the body of patient 12 that function to drive fluid from expandable balloon 22 to waste storage structure 26. In one example, pressure from fresh ablation reagents entering expandable balloon 22 (e.g., before or after mixing) may exhaust waste fluids in the balloon through an exhaust lumen of catheter 18. A user may actuate actuatable trigger 24, causing ablation reagents 14 to enter expandable balloon 22. As the ablation reagents enter the expandable balloon, the pressure of the reagents can force waste fluid in balloon out through an exhaust lumen of catheter 18. In another example, delivery device 16 or another device coupled to catheter 18 may be configured to generate a vacuum that draws waste fluid in expandable balloon 22 out of patient 12 and into waste storage structure 26. The vacuum pressure can be applied at or adjacent proximal end 20A of catheter 18, causing the fluid in expandable balloon 22 to be drawn out of the balloon through a lumen of catheter 18 and into waste storage structure 26.

In the example of FIG. 1A, delivery device 16 includes a syringe 30 that can be used to generate a vacuum for drawing waste fluid out of expandable balloon 22 and into waste storage structure 26. Syringe 30 may be coupled (e.g., mechanically and/or electrically) to actuatable trigger 24 so that actuation of the trigger retracts a plunger through a barrel of the syringe, generating a vacuum that draws fluid out of expandable balloon. For example, when delivery device 16 is configured as shown in FIG. 1A, a user can apply a force to actuatable trigger 24 to depress the trigger. Depressing actuatable trigger 24 may extend plunger 25 through reservoirs housing ablation reagents 14. Depressing actuatable trigger 24 may also extend a plunger through a barrel of the syringe 30. To avoid injecting fluid (e.g., air) in syringe 30 into patient 12 as the syringe plunger extends through the barrel of the syringe, delivery device 16 may include a diverter valve or other mechanism that prevents air from within the syringe from entering catheter 18. Upon releasing actuatable trigger 24, the plunger of syringe 30 may automatically refract, generating a vacuum that draws fluid out of expandable balloon 22 and into waste storage structure 26.

When delivery device 16 is configured to exhaust waste fluid from expandable balloon 22, the device may or may not include features to control the rate at which waste fluid is withdrawn from the balloon. In some embodiments, the waste fluid is withdrawn from the balloon in response to a pressure of the balloon. For example, a valve device, such as a pressure valve or a check valve, can be included. In such embodiments, after the balloon reaches a designated pressure, waste fluid will begin to exhaust from the balloon and will continue to exhaust as additional reactants and/or products of reactants are delivered into the balloon. In certain embodiments, during a treatment procedure reactants and/or products of reactants are continuously delivered into a balloon and waste is continuously exhausted. Embodiments with controlled exhaust rates are useful for maintaining balloon conformality with a body lumen during a procedure.

In general, ablation system 10 defines a closed system in which ablation reagents 14 are injected to patient 12 and waste fluid is evacuated out of the patient without direct contact with bodily tissue, either continuously or in batch. Heat generated by an exothermic reaction between ablation reagents 14 can be transmitted through a fluid impermeable wall of expandable balloon 22, preventing the fluids in the balloon from directly contacting tissue.

FIG. 3 is a functional block diagram illustrating components of an example ablation system 10 where delivery device 16 is capable of operating under instructions stored on a computer readable medium. The example system includes a processor 50, memory 51, a fluid delivery pump 52, a first fluid reservoir 54, a second fluid reservoir 56, a third fluid reservoir 58, a vacuum generator 60, a power source 62, and a sensor 64. Processor 50 is communicatively coupled to memory 51, fluid delivery pump 52, and vacuum generator 60. Processor 50 may also be communicatively coupled to sensor 64. Fluid delivery pump 52 may be connected to first fluid reservoir 54, second fluid reservoir 56, and third fluid reservoir 58 through fluid pathways. First fluid reservoir 54, a second fluid reservoir 56, a third fluid reservoir 58 are in fluid communication with catheter 18. Vacuum generator 60 is in fluid communication with both catheter 18 and conduit 28. Power source 62 delivers operating power to various components of delivery device 16 and, optionally, sensor 64. Optionally, power source 62 can include a battery. Such embodiments are useful for providing a portable ablation system 10 that is not required to be connected to a power grid to be used in a procedure, as is required with RF ablation systems.

During operation of delivery device 16, processor 50 controls fluid delivery pump 52 with the aid of instructions stored in memory 51 to deliver one or more fluids stored in reservoirs 54, 56, and 58 to expandable balloon 22 via catheter 18. Instructions executed by processor 50 may, for example, define the rate and/or amount of fluid that is delivered to expandable balloon 22 within patient 12 from each of first fluid reservoir 54, second fluid reservoir 56, and/or third fluid reservoir 58. The instructions may further control the timing, rate, and/or operation of vacuum generator 60 for withdrawing fluid from expandable balloon 22 via catheter 18. Processor 50 may receive user input (e.g., via a user interface not shown) to initiate operation of delivery device 16, refill fluid reservoirs, change fluid delivery characteristics, or the like.

First fluid reservoir 54, second fluid reservoir 56, and/or third fluid reservoir 58 may house the same fluid, e.g., in different quantities of or different concentrations, to provide flexibility for controlling the amount of heat generated by the exothermic reaction within ablation system 10. Alternatively, at least one of first fluid reservoir 54, second fluid reservoir 56, and third fluid reservoir 58 may house a fluid different than at least one of other of first fluid reservoir 54, second fluid reservoir 56, and third fluid reservoir 58. In some examples, at least one of reservoirs 54, 56, and 58 houses first ablation reagent 14A (FIG. 1A) while at least one of the other reservoirs houses second ablation reagent 14B. In these examples, the third reservoir may house an additional reagent, which may or may not be the same as one of ablation reagents 14, or the reservoir may house a non-reagent fluid such as saline or water. When ablation system 10 includes a reservoir that houses a non-reagent fluid, the non-reagent fluid may be delivered to expandable balloon 22 prior to delivering reagents 14 to the expandable balloon. This may allow the user to confirm the fluid integrity of the ablation system, clear air bubbles, fill the balloon to expand the balloon within a lumen of patient 12, or perform other functions.

In general, first fluid reservoir 54, second fluid reservoir 56, and third fluid reservoir 58 may be arranged in numerous locations within delivery device 16 including, e.g., in a stacked arrangement (e.g., one on top of another) or in a coplanar arrangement (e.g., side-by-side). In some examples, one or more of fluid reservoirs 54, 56, and 58 are separate from delivery device 16 rather than contained within at least a portion of the device. Fluid reservoirs 54, 56, and 58 may or may not be replaceable. Example replaceable reservoirs include bags, cartridges, and syringes. Further, while ablation system 10 in the example of FIG. 3 includes three reservoirs, in other examples, the system may include fewer reservoirs (e.g., one or two reservoirs) or more reservoirs (e.g., four, five, or more).

Delivery device 16 in the example of FIG. 3 includes fluid delivery pump 52. Fluid delivery pump 52 can be any mechanism that delivers fluid from fluid reservoirs 54, 56, and 58 to a target ablation site within patient 12 via catheter 18. In various examples, fluid delivery pump 52 may be an axial pump, a centrifugal pump, or a piston-driven pump. In one example, fluid delivery pump 52 comprises a plurality of pistons, one piston being associated with each of fluid reservoirs 54, 56, and 58. Under the control of processor 50, fluid delivery pump 52 may extend a piston associated with a particular reservoir into the particular reservoir to pressurize a fluid and discharge it into catheter 18.

When configured with vacuum generator 60, the vacuum generator may generate a vacuum in response to instructions received from processor 50 to draw waste fluid out of expandable balloon 22 and into waste storage structure 26. In some examples, vacuum generator 60 includes a syringe with a plunger that retracts under the control of processor 50 to generate a vacuum for exhausting expandable balloon 22. In other examples, vacuum generator 60 may be a vacuum pump. Other types of vacuum generators are possible.

In general, awareness of different properties within delivery device 16, catheter 18, and/or expandable balloon 22 including, e.g., temperatures, pressures, volumes, and the like, may be desirable to monitor the operation of ablation system 10. Consequently, ablation system 10, in various examples, may include at least one sensor 64. In the example of FIG. 3, sensor 64 is positioned within expandable balloon 22. Sensor 64 may be communicatively coupled to processor 50, e.g., via a wired connection extending through catheter 18, or via a wireless connection. Other example locations for a sensor in addition to or in lieu of sensor 64 include within a lumen (e.g., all lumens) of catheter 18, within a mixing zone of catheter 18 when the catheter is configured with a mixing zone, or within delivery device 16 proximate catheter 18. In still another example, sensor 64 may be positioned outside of expandable balloon 22, e.g., to detect a temperature of an external surface of expandable balloon 22 and/or a temperature of tissue being ablated by ablation system 10.

Sensor 64 can be configured to detect any suitable characteristic including, e.g., temperature, pressure, and/or fluid flow. In various examples, sensor 64 may be a pressure sensor, a flow sensor, a capacitive sensor, an acoustic sensor, an optical sensor, or a combination of types of sensor. During operation, processor 50 may receive a signal generated by sensor 64 and analyze the signal with reference to memory 51. Based on the analysis, processor 50 may control fluid delivery pump 52, vacuum generator 60, or other aspects of delivery device 10 to vary the quantity or make up of fluid entering or leaving patient 12.

In one example, sensor 64 includes (e.g., is) a temperature sensor that is communicatively coupled to processor 50. In response to signals received by sensor 64, processor 50 can determine a temperature within a mixing zone of catheter 18 (when so configured), within expandable balloon 22, of waste fluid returning to delivery device 16 from expandable balloon 22, of tissue at a target ablation site within patient 18, and/or at other locations within ablation system 10. Based on the determined temperature, processor 50 can control delivery device 16 to perform a variety of functions.

In one example, processor 50 may compare the determined temperature with data stored in memory 51 to confirm or validate that an exothermic reaction is taking place. Processor 50 may compared the determined temperature to one or more thresholds (e.g., a body temperature of patient 12, a temperature of ablation reactions 14 as the reactants enter catheter 18, or above one or more other thresholds, such as 45 degrees Celsius, 50 degrees Celsius, 90 degrees Celsius, etc.) and determine, based on the comparison, whether an exothermic reaction is taking place. This can help ensure the integrity and intended operation of ablation system 10.

In response to the comparison, processor 50 may control delivery device 16 to notify a user of an operational status of the system (e.g., when the device includes a user interface), and/or control fluids entering or leaving patient 12. For example, processor 50 may control fluid delivery pump 52 to vary the rate at which one or more fluids are delivered into catheter and/or the source of fluid being delivered into the catheter, e.g., by selectively delivering fluid from first fluid reservoir 54, second fluid reservoir 56, or third fluid reservoir 58, so as to control the temperature within expandable balloon 22 and/or the rate at which a temperature in the balloon increases or decreases. As another example, processor 50 may control vacuum generator 60 to exhaust fluid from expandable balloon 22, e.g., if the fluid in the balloon is above a threshold temperature. In this manner, ablation system 10 may control temperature within expandable balloon 22 and/or the rate at which a temperature in the balloon increases or decrease. Controlling the temperature of expandable balloon 22 during ablation within patient 12 may help provide a more uniform tissue ablation than if the temperature is not actively controlled.

In another example, sensor 64 includes (e.g., is) a pressure sensor that is communicatively coupled to processor 50. When the pressure sensor is positioned detect a pressure within expandable balloon 22 as shown in the example of FIG. 1A, processor 50 can determine a pressure within the balloon in response to signals received from the sensor. Processor 50 may compare the determined pressure to data stored in memory 51 and control the operation of delivery device 16 based on the comparison. If the pressure within expandable balloon is not correct, processor 50 can, e.g., control vacuum generator 60 to remove at least some (and in some cases all) of the fluid with the balloon to reduce the pressure of the balloon.

In examples in which ablation system 10 includes sensor 64, the sensor can be a power or non-powered sensor. An example of a non-powered sensor is a non-powered temperature sensor, such as a temperature sensitive material (e.g., a thin-film temperature band or other temperature material coupled to the exhaust line). Other non-powered sensors are available, as will be appreciated by those of skill in the art.

Processor 50 may include any one or more of a microprocessor, a controller, a digital signal processor (DSP), an application specific integrated circuit (ASIC), a field-programmable gate array (FPGA), or equivalent discrete or integrated logic circuitry. In some examples, processor 50 may include multiple components, such as any combination of one or more microprocessors, one or more controllers, one or more DSPs, one or more ASICs, or one or more FPGAs, as well as other discrete or integrated logic circuitry. The functions attributed to processor 50 herein, may be embodied as software, firmware, hardware or any combination thereof.

Under the control of processor 50, delivery device 16 may be configured to perform functions other than those specially described above. In one example, processor 50 may be configured to control fluid delivery pump 52 to clear gas bubbles from catheter 18 and expandable balloon 22, e.g., by injecting saline or another non-reagent fluid into the catheter prior to delivering ablation reagents 14 to the catheter. In another example, delivery device 16 may include one or more sensors for monitoring the quantity of liquid in first fluid reservoir 54, second fluid reservoir 56, and/or third fluid reservoir 58. In this example, processor 50 can monitor the quantity of liquid in the reservoirs, e.g., to ensure a proper supply of fluid(s) for ablation therapy. Processor 50 may also control a user interface (e.g., when the device includes a user interface) to notify a user of the quantity of liquid on a specific reservoir, the type of liquid in a specific reservoir, the need to replace or refill a specific reservoir, or the like. In still another example, delivery device 16 may be configured to control retraction of catheter 18 from within patient 12 and/or confirm the success and consistency of the retraction.

In general, memory 51 stores instructions and related data that, when executed by processor 50, cause delivery device 16 and processor 50 to perform the functions attributed to them in this disclosure. Memory 51 may comprise a non-transitory computer-readable medium such as, e.g., random access memory (RAM), read only memory (ROM), programmable read only memory (PROM), erasable programmable read only memory (EPROM), electronically erasable programmable read only memory (EEPROM), flash memory, a hard disk, or other computer readable media. Instructions embedded or encoded in a computer-readable storage medium may cause a programmable processor, or other processor, to perform methods, techniques, and actions described in this disclosure, e.g., when the instructions are executed.

FIG. 4A is a diagram illustrating an example expandable balloon 22 connected to an example catheter 18 that may be used in ablation system 10 (FIGS. 1A and 3). FIG. 4B is a cross-sectional illustration of the expandable balloon 22 shown in FIG. 4A, taken along the A-A line shown on FIG. 4A. Expandable balloon 22 is positioned adjacent distal end 20B of catheter 18, which provides a fluid connection between an interior space defined by the expandable balloon and outside of the body of patient 12 (FIG. 1A). Expandable balloon 22 is defined by a wall 70 that, in some examples, is constructed of a fluid impervious biocompatible material such as a biologically inert polymer. Examples include polyethylene and nylon.

Expandable balloon 22 can be inserted into a lumen of patient 12 such as airway of the patient to thermochemically ablate tissue in or adjacent the lumen. In general, expandable balloon 22 may be inserted into patient 12 in a deflated state, e.g., without fluid in the balloon so the balloon is at least partially (and in some cases fully) collapsed. Once in a target position within patient 12 for ablating tissue, the balloon can be expanded by injecting pressurized fluid into the balloon via catheter 18. The pressurized fluid may be a non-reagent fluid such as, e.g., saline or water. The pressurized fluid may also be an ablation reagent fluid or a mixture or reaction product of ablation reagent fluids. In some examples, expandable balloon 22 includes a steering member extending from the distal end of the balloon and configured to guide the balloon to a desired target location. The steering member may deflect a distal tip of the balloon in a desired direction to navigate to a particular body lumen, e.g., a particular bronchi or bronchiole.

Expandable balloon 22 is configured to ablate tissue by transferring thermal energy generated from an exothermic reaction to tissue located adjacent an external surface of wall 70 of the balloon. For this reason, expandable balloon 22 may be configured to expand (e.g., from a deflated state to an expanded state) until the balloon conforms to a body lumen of patient 12 into which the balloon is inserted. In some examples, expandable balloon 22 is configured to conform to a body lumen by expanding until the balloon substantially matches a size and/or shape (e.g., in the X-direction indicated on FIG. 4A) of the lumen into which the balloon is inserted along the balloon's working length. For example, expandable balloon 22 may be configured to expand until wall 70 of balloon is in direct contact with at least a portion of a wall of the lumen into which the balloon is inserted. In some additional examples, expandable balloon 22 may be configured to expand until the balloon directly contacts a wall of the lumen into which the balloon is inserted about substantially an entire perimeter (e.g., circumference) of the balloon. In some embodiments, contact with the patient's anatomical structure will cause the expandable balloon to assume a non-uniform expanded shape (e.g., circumferentially and/or longitudinally). Conforming expandable balloon 22 to the body lumen of patient 12 may increase the efficiency with which thermal energy is transferred from the balloon to the tissue of patient 12, thereby increasing the rate and/or amount of tissue ablated. Further, conforming the expandable balloon to the airway around its entire circumference occludes the airway, which may allow for lower treatment temperatures because it slows heat dissipation.

In the example of FIGS. 4A and 4B, expandable balloon 22 defines an elongated member that is longer along a major axis (i.e., in the Z-direction indicated on FIG. 4A) than an axis orthogonal to the major axis (e.g., the X-direction or the Y-direction indicated on FIG. 4B). Further, expandable balloon 22 defines circular cross-sectional shape (i.e., in the Y-X plane indicated on FIG. 4B). Expandable balloon 22 can define shapes other than the shape illustrated in FIGS. 4A and 4B. For example, expandable balloon 22 can define any polygonal (e.g., square, hexagonal) or arcuate (e.g., circular, elliptical) shape, or even combinations of polygonal and arcuate shapes. The specific shape of expandable balloon 22 may vary, e.g., based on the shape of the lumen of patient 12 into which the balloon is intended to be inserted.

Further, the specific size of expandable balloon may vary, e.g., based on the size of the lumen of patient 12 into which the balloon is intended to be inserted and/or the size of the area with patient 12 to be ablated. As described above, the techniques, devices, and systems described in this disclosure may be used to treat pulmonary disorders such as asthma. For these applications, expandable balloon 22 may be sized to conform to a human airway such as, e.g., a lumen of the bronchial tree. Although expandable balloon 22 can be inserted into patient 12 without using a bronchoscope, in some examples, the balloon may be inserted into the patient through a lumen of a bronchoscope. In these examples, the expandable balloon may be sized to fit through a working channel or lumen of the bronchoscope (e.g., in a deflated state). For instance, when used with a bronchoscope having a channel or lumen with a cross-section dimension (e.g., diameter) of 2 mm, expandable balloon 22 may have a cross-sectional size (i.e., in the Y-X plane indicated on FIG. 4B) less than approximately 2 mm.

Expandable balloon 22 may have any suitable dimensions. That being said, in some examples, the balloon may define a major length (i.e., in the Z-direction indicated on FIG. 4A) ranging from approximately 5 millimeters (mm) to approximately 40 mm (e.g., approximately 10 mm to approximately 20 mm), and a major width (i.e., outer diameter in the X-direction or Y-direction indicated on FIG. 4B) ranging from approximately 1.5 mm to approximately 3 mm in a deflated state and from approximately 2 mm to approximately 15 mm in a fully inflated state. Such an example expandable balloon may be sized to fit within a bronchi or bronchiole of patient 12. In some examples, expandable balloon 22 may have a size ranging from 7 Fr to 9 Fr. The foregoing dimensions are merely examples, however, and other dimensions are both contemplated and possible.

Further, a kit may be provided with several balloons sized to treat certain sizes of body lumens (e.g., an airway lumen segment with an inner diameter of between approximately 3 mm and approximately 12 mm and a segment length of between approximately 10 mm and approximately 25 mm). Each balloon in the kit, and its associated catheter, can be easily connected to the delivery device during a treatment. For example, a first balloon with a major width of approximately 1.5 mm to approximately 3 mm and a length of approximately 5 mm to approximately 15 mm in an inflated state may first be used to treat fifth to seventh generational branches of the airway. The first balloon and its associated catheter can then be disconnected from the delivery device and a second balloon with a larger major width and, optionally a longer length, can be used to treat, e.g., third to fifth generation branches of the airway. The procedure can continue iteratively in this manner until an nth balloon having a major width of approximately 12 mm to approximately 15 mm and, optionally, a length of between approximately 15 mm and approximately 30 mm and is used to treat the largest diameter portions of the airway.

During operation of ablation system 10, expandable balloon 22 receives ablation reagents 14 via catheter 18. As described above, catheter 18 can have a single lumen or multiple lumens. In the example of FIG. 4A, catheter 18 includes a first lumen 72 in fluid communication with a reservoir housing a first ablation reagent, a second lumen 74 in fluid communication with a reservoir housing a second ablation reagent, and a third lumen 76 for exhausting fluid from the balloon (e.g., to waste storage structure 26 in FIG. 1A). Exothermic reaction of the ablation reagents generates heat that can conduct through wall 70 of the balloon so as to heat an exterior surface of the wall for ablating tissue within patient 12.

In some examples, ablation system 10 is configured to combine ablation reagents for generating an exothermic reaction outside of expandable balloon and deliver the combined reagents (or a reaction product thereof) to expandable balloon 22. When so configured, the ablation reagents may be combined within delivery device 16 or within catheter 18 (e.g., between proximal end 20A and distal end 20B) and subsequently delivered under pressure to expandable balloon 22.

In some examples, as shown in FIG. 4C, catheter 18 may include a mixing zone 80 positioned proximally of expandable balloon 22 (e.g., between proximal end 20A and distal end 20B, including outside of a patient's body in use during a procedure). The mixing zone may or may not be an area of larger cross-sectional area compared to other cross-sectional areas along the length of the catheter. In such examples, the ablation reagents may be separately delivered and combined within the mixing zone and then subsequently delivered under pressure to expandable balloon 22. As shown in FIG. 4C, in some embodiments catheter 18 is provided with a first lumen 72 for delivering a first ablation reagent and a second lumen 74 for delivering a second ablation reagent. The lumens combine into a common lumen 75 at a mixing zone 80 somewhere along the catheter's length, including at a location outside of a patient's body during a procedure. The mixed ablation reagents are then delivered to the expandable balloon via the common lumen. In some embodiments the mixing zone includes structure to promote mixing of the ablation reagents, such as baffles.

In the example of FIG. 4A, expandable balloon 22 includes a mixing zone 80 that is positioned at least partially, and in the illustrated example fully, within an interior space defined by the balloon itself. Mixing zone 80 is fluidly connected to the first lumen 72 in fluid communication with a reservoir housing a first ablation reagent and second lumen 74 in fluid communication with a reservoir housing a second ablation reagent. Mixing zone 80 includes a surface 81 (e.g., a cylindrical surface) with at least one aperture which, in the case of FIG. 4A, is illustrated as a plurality of apertures 82 for communicating fluid between an interior of the mixing zone and an area defined between the mixing zone and wall 70 of expandable balloon 22. Mixing zone 80 may be a defined space and/or structure within expandable balloon 22 for receiving ablation reagents and promoting efficient mixing of the reagents, and efficient dispersing of the reagents, or a reaction product thereof, into the expandable balloon. Apertures 82 may be positioned to substantially evenly disperse the different ablation into the mixing zone to provide substantially uniform mixing of the reagents, which, in turn, may provide substantially uniform heating along the length of the balloon. In other examples, expandable balloon 22 does not include mixing zone 80. In such embodiments, for example, the first and second reagents are directly injected into the interior of the expandable balloon or mixed along the length of the catheter.

In some embodiments the surface 81 with at least one aperture 82 is provided as shown in FIG. 4A in combination with a mixing zone located along the length of the catheter as shown in FIG. 4C. In such embodiments, the surface and aperture is useful for uniformly dispersing the mixed ablation reagent, or the reaction product thereof, into the expandable balloon.

As described above with respect to FIGS. 1A and 3, ablation system 10 may be configured to exhaust fluids from expandable balloon 22, e.g., to deflate the balloon and/or reduce the temperature in the balloon. For this reason, catheter 18 may include an exhaust lumen extending from expandable balloon 22 to outside the body of patient 12. In the example of FIG. 4A, catheter 18 includes third lumen 76 for exhausting fluid from the balloon (e.g., to waste storage structure 26 in FIG. 1A). In different examples, expandable balloon 22 and/or catheter 18 can includes a single exhaust, a plurality of exhausts, or not exhausts whatsoever. In some examples, third lumen 76 can include metered slots, e.g., to help keep an airway or bodily lumen from collapsing the balloon and trapping hot fluid at a distal end of the balloon. Further, these slots, which can also be referred to as baffles, can restrict flow of the exhaust fluids in the exhaust lumen to help maintain pressure in the balloon during a procedure.

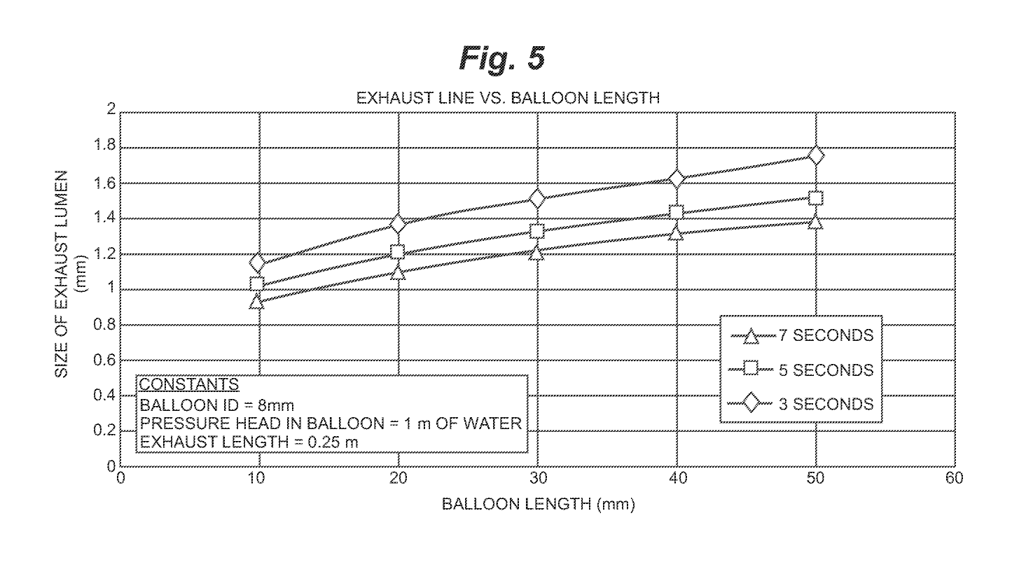

In instances in which ablation system 10 includes a third lumen 76 for exhausting fluid from the balloon, the lumen can have any suitable size and shape. FIG. 5 illustrates generally an example of a relationship between a size of an exhaust lumen and balloon length, showing exemplary times to remove the thermal fluid from the balloon catheter under various conditions.

The techniques, devices, and systems described in this disclosure can be used to treat a variety of symptoms and disorder including, e.g., pulmonary disorders such as asthma. Asthma is typically characterized as a chronic inflammatory process in the airway, causing increasing the resistance to airflow within the lungs. Many cells and cellular elements may be involved in the inflammatory process, particularly mast cells, eosinophils T lymphocytes, neutrophils, epithelial cells, and even airway smooth muscle itself. The reactions of these cells can result in an associated increase in the existing sensitivity and hyper-responsiveness of the airway smooth muscle cells that line the airways. Hyper-responsiveness can be evaluated with several chemical challenges such as methacholine and the inhalation of hypertonic saline. Both have already been widely used to induce sputum and to collect inflammatory cells and cytokines in asthmatics.

The chronic and persistent nature of severe asthma can also lead to remodeling/structural changes of the airway wall, which can further affect the function of the airway wall and influence airway hyper-responsiveness. Severe asthma can also include excess mucus production, plugging and epithelial denudation. In susceptible individuals, asthma symptoms may include recurrent episodes of shortness of breath (dyspnea), wheezing, chest tightness, and cough.

Thermochemical ablation of smooth muscle tissue (and/or other tissues) lining the airway wall of an asthmatic patient may help alleviate asthmatic symptoms. Thermochemical ablation accomplished by ablation system 10 may shrink or destroy airway wall tissue, enlarging the airway to improve air flow through the airway. Thermochemical ablation accomplished by ablation system 10 may also shrink or destroy tissue responsible for contracting (e.g., smooth muscle) during an asthmatic attack, minimizing or eliminating airway constriction attendant to muscle contraction. Further, heat from the ablation may also cross-link sub-mucosa and/or collagen to stiffen the airway.

To thermochemically ablate tissue in the airway of an asthmatic patient, an expandable balloon coupled to a catheter can be inserted into an airway of the patient and expanded to conform to the wall of the airway along a portion of its length. The expandable balloon can be heated by an exothermic reaction generated by combining two or more ablation reagents together. The exothermic reaction can take place entirely within the expandable balloon, partially within the expandable balloon, within the catheter, or external to the body prior to delivering the fluid to the catheter connected to the expandable balloon. Independent of where the exothermic reaction takes place, the heated byproduct from the reaction can heat an external wall of the expandable balloon to a temperature appropriate for thermal ablation/thermoplasty of the airway wall, e.g., while keeping surrounding tissues at temperatures below which could cause permanent injury and severe pain. In some examples, the thermal energy applied to the airway wall is applied for a limited duration, e.g., such that the endothelium and smooth muscle layers of the airway are heated to a temperature sufficient to result in their eventual obliteration while not raising the temperature in the surrounding tissues beyond the adventitia and parenchyma to injury levels. In this way, heat can be applied to the airway(s) of an asthmatic patient to obliterate tissue responsible for contracting during an asthmatic attack. The heat may also cause other physiological responses which result in increased airway diameters or reduced sensitivity to asthmatic attacks.

When inserting the expandable balloon into a patient suffering from asthma, the expandable balloon can be inserted to any suitable position within the airway system of the patient. In humans, the trachea (windpipe) conducts inhaled air into the lungs through its tubular branches, called bronchi. The bronchi then divide into smaller and smaller branches (bronchioles). Specifically, the right and left primary bronchi each branch into lobar/secondary bronchi (one to each lobe of the lung—thus, two on the left and three on the right), then divide again into segmental/tertiary bronchi and finally terminal bronchioles, which lead to alveolar sacs. In different examples in accordance with the disclosure, the expandable balloon may be inserted and heated so as to ablate tissue in the trachea of a patient, a primary bronchi of the patient, a secondary bronchi of the patient, a tertiary bronchi of the patient, a terminal bronchiole of the patient, or a combination thereof. In certain embodiments, the furthest (and smallest) branches accessible with the ablation system will be treated first, and the practitioner will work backwards towards the mouth treating closer (and larger) portions of the airway. As described herein, different sized balloons can be used to treat different portions of the airway system. In some embodiments, substantially the entire length of the airway is treated from the fifth to seventh (e.g., sixth) generation branches to the largest bronchi, with minimal longitudinal gaps between treatment areas.

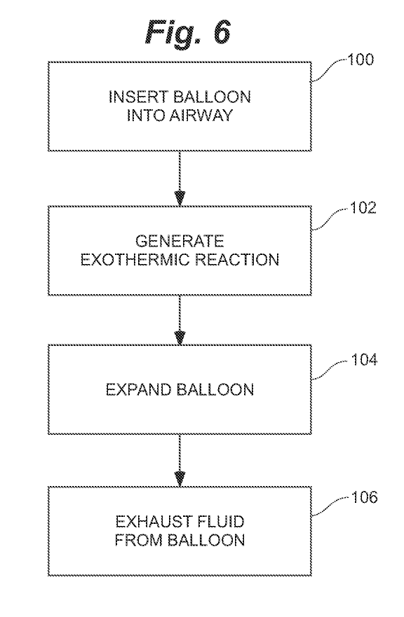

Different thermochemical ablation systems, devices, and techniques have been described in relation to FIGS. 1A-5. FIG. 6 is a flow chart illustrating an example method for thermochemically ablating tissue in an airway using thermochemical ablation reagents and expandable balloon. For ease of description, the method of FIG. 6 is described as executed by ablation system 10 and delivery device 16 (FIG. 1A). In other examples, however, the method of FIG. 6 may be executed by systems and devices having other configurations, as described herein.

As shown in FIG. 6, expandable balloon 22 adjacent distal end 20B of catheter 18 can be inserted into an airway of patient 12 (100). An exothermic reaction can be generated by combining ablation reagents 14 to transfer heat to an external surface of the expandable balloon (102). Further, expandable balloon 22 can be inflated to conform to the airway of the patient into which the expandable balloon is inserted (104). In some examples, expandable balloon 22 is expanded by introducing combined or individual ablation reagents under pressure into the balloon. In other examples, a non-ablation reagent (e.g., saline) is introduced into the balloon to expand the balloon. Therefore, although generating an exothermic reaction (102) and expanding expandable balloon 22 (104) are shown as separate steps, in other examples, the steps may occur substantially simultaneously. Further, additional reagents or the reaction product thereof may be continuously delivered to the expandable balloon to maintain a desired pressure and/or temperature within the balloon. After suitable ablating the tissue adjacent the expanded balloon, fluid from within the balloon is exhausted to deflate the balloon (106).

The technique of FIG. 6 includes inserting expandable balloon 22 connected to catheter 18 into an airway of patient 12 (100). The expandable balloon and/or catheter may be prepared prior to inserting the devices into the patient. For example, expandable balloon 22 may be de-aired, e.g., by injected saline through the balloon, and/or pressure tested to validate the integrity of the balloon. Further, when catheter 18 includes a guide wire lumen, the lumen may be flushed, e.g., with saline, to clear the lumen to receive the guide wire. Additionally, proximal end 20A of catheter 18 can be attached to delivery device 16 to establish fluid communication between expandable balloon 22 and reservoirs housing first ablation reagent 14A and second ablation reagent 14B.

In some examples, expandable balloon 22 and catheter 18 are inserted directly into the airway of the patient (100) without the aid of an access device. In other examples, an access device is first inserted to provide access to a portion of the airway and the expandable balloon and catheter are inserted through the access device. A bronchoscope is an example of an access device.

Expandable balloon 22 is inserted to a target ablation location within the airway. External imaging devices may be used to identify the target location and to help the user insert the balloon to the location. Example imaging devices include, but are not limited to, ultrasound, direct vision, and fluoroscopy. In different examples, the user can insert the expandable balloon so the balloon is positioned in the trachea of a patient, a primary bronchi of the patient, a secondary bronchi of the patient, a tertiary bronchi of the patient, or a terminal bronchiole of the patient. Regardless of the target location, the user may insert the expandable balloon so the distal end of the balloon is adjacent the most cranial part of the airway to be treated.

The technique of FIG. 6 includes generating an exothermic reaction by combining first ablation reagent 14A with second ablation reagent 14B (102). A user may apply a physical force to actuatable trigger 24, actuating the trigger and causing plunger 25 to push first ablation reagent 14A and second ablation reagent 14B out of reservoirs housing the reagents and into catheter 18. In some examples, first ablation reagent 14B and second ablation reagent 14B are pressurize at substantially the same time upon actuating actuatable trigger 24, causing the reagents to be delivered substantially simultaneously to catheter 18. In other examples, first ablation reagent 14B and second ablation reagent 14B may be delivered at different times and/or different rates upon actuating actuatable trigger 24, causing the reagents to be delivered at different times and/or different rates to catheter 18.

Upon releasing first ablation reagent 14A and second ablation reagent 14B from their respective reservoirs, the ablation reagents may combine in any suitable location within ablation system 10 to generate heat associated with an exothermic reaction. In some examples, first ablation reagent 14A and second ablation reagent 14B combine within delivery device 16 or a mixing zone located proximally of expandable balloon 22 and the combined reagents (or a reaction product thereof) are delivered to expandable balloon 22. In other examples, first ablation reagent 14A and second ablation reagent 14B combine within expandable balloon 22.

Thermochemical ablation of a portion of an airway according to FIG. 6 also includes expanding expandable balloon 22 to conform to the airway of the patient into which the expandable balloon is inserted (104). Conforming expandable balloon 22 to the airway may increase the efficiency with which thermal energy is transferred from the balloon to the tissue of patient 12.

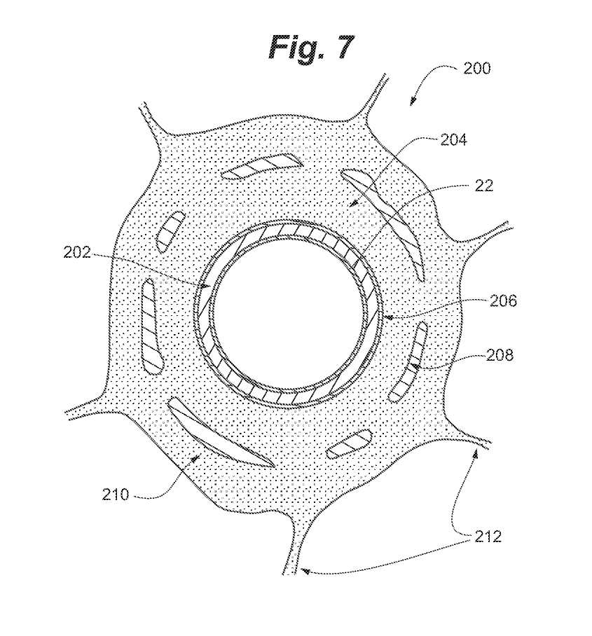

In some examples, expandable balloon 22 is expanded until the balloon substantially matches a size and/or shape of the airway into which the balloon is inserted. For example, expandable balloon 22 may expand until an external surface or wall of balloon is in direct contact with at least a portion of a wall of the airway into which the balloon is inserted. FIG. 7 shows an example of a expandable balloon 22 conformal with an airway 200 around its circumference, thereby occluding the airway. Airway 200 includes an epithelium 202, internal submucosa 204, subepithilial collagen 206, smooth muscle 208, adventitia 210, and parenchymal attachments 212. FIG. 7 depicts the outer surface of the expandable balloon in apposition with the epithelium 202 of the airway.

Expandable balloon 22 may expand to conform to the airway (104) in response to pressurized fluid entering an interior of the expandable balloon. In some examples, a non-ablation reagent, e.g., saline, may be injected into the balloon via catheter 18 so as to expand the balloon. In other examples, individual or combined ablation reagents (e.g., a reaction product thereof) can be delivered under pressure to expandable balloon 22 to expand the balloon. In either set of examples, heated fluid with expanded balloon 22 can heat an external wall of the expandable balloon to a temperature sufficient for thermal ablation/thermoplasty of the airway wall, e.g., while keeping surrounding tissues at temperatures below which could cause permanent injury and severe pain. In some examples, the thermal energy applied to the airway wall is applied for a limited duration sufficient to obliterate the endothelium and smooth muscle layers of the airway while not raising the temperature in the surrounding tissues beyond the adventitia and parenchyma to injury levels.

After suitably ablating the tissue adjacent the expanded expandable balloon 22, fluid from within the balloon is exhausted to deflate the balloon (106). Upon releasing actuatable trigger 24, a plunger within syringe 30 may retract, generating a vacuum that withdraws fluid out of expandable balloon 22 and into waste storage structure 26. During or after exhausting expandable balloon 22, fresh ablation reactants, or the reaction product thereof, can be injected into the balloon to further heat the same region of the airway of patient 12 initially heated. Alternatively, expandable balloon 22 can be retracted or inserted deeper into the airway of the patient for further ablation of the airway structure, or the balloon may be retracted entirely from within the patient. In some embodiments, substantially the entire length of the airway is treated from the fifth to seventh (e.g., sixth) generation branches of the airway having an inner diameter of approximately 1 mm to approximately 3 mm to the largest bronchi having a diameter of approximately 10 mm to approximately 20 mm, with minimal longitudinal gaps between treatment areas. In certain embodiments, the furthest (and smallest) branches accessible with the ablation system will be treated first, and the practitioner will work backwards towards the mouth treating closer (and larger) portions of the airway, optionally using different sized balloons to treat different portions of the airway. In some embodiments, substantially the entire length of the airway is treated from the fifth to seventh (e.g., sixth) generation branches to the largest bronchi, with minimal longitudinal gaps between treatment areas.

Various examples have been described. These and other examples are within the scope of the following claims.

1. An ablation system comprising:
a first reservoir configured to house a first ablation reagent;
a second reservoir configured to house a second ablation reagent, the first and second ablation reagents being configured to generate an exothermic reaction when mixed; and
a catheter configured to be inserted into at least a portion of a human airway, the catheter extending from a proximal end to a distal end and including an expandable balloon adjacent the distal end, the catheter being configured to provide fluid communication between the first reservoir, the second reservoir, and an interior of the expandable balloon.

2. The ablation system of claim 1, wherein the expandable balloon is configured to be inserted into at least a portion of a bronchial tree and the expandable balloon is configured to expand so as to conform to the portion of the bronchial tree into which the expandable balloon is inserted.

3. The ablation system of claim 2, wherein the expandable balloon is configured to be inserted into at least one of a primary bronchi, a secondary bronchi, a tertiary bronchi, and a terminal bronchiole.

4. The ablation system of claim 2, wherein the expandable balloon is configured to conform to the portion of the bronchial tree into which the expandable balloon is inserted by at least expanding until the expandable balloon contacts a wall of the bronchial tree about substantially an entire perimeter of the expandable balloon.

5. The ablation system of claim 1, wherein the exothermic reaction is configured to cause an exterior surface of the expandable balloon to increase in temperature to a temperature sufficient to ablate tissue of the human airway into which the expandable balloon is inserted.

6. The ablation system of claim 1, wherein the exothermic reaction is configured to cause an exterior surface of the expandable balloon to increase to a temperature ranging from approximately 45 degrees Celsius to approximately 75 degrees Celsius for a period of at least 5 seconds.

7. The ablation system of claim 1, wherein the first ablation reagent is selected from the group consisting of HCl, AcOH, and citric acid, and the second ablation reagent is an alkali metal hydroxide.

8. The ablation system of claim 1, wherein the catheter comprises a first lumen configured to provide fluid communication between the first reservoir and the interior of the expandable balloon and a second lumen configured to provide fluid communication between the second reservoir and the interior of the expandable balloon.

9. The ablation system of claim 8, wherein the catheter further comprises a mixing zone positioned proximally of the expandable balloon and in fluid communication with the expandable balloon, wherein the first lumen is configured to provide fluid communication between the first reservoir and the mixing zone and the second lumen is configured to provide fluid communication between the second reservoir and the mixing zone, and a common lumen is configured to provide fluid communication between the mixing zone and the expandable balloon.

10. The ablation system of claim 8, wherein the catheter further comprises an exhaust lumen separate from the first lumen and the second lumen, the exhaust lumen being configured to exhaust a reaction product of the first ablation reagent and the second ablation reagent from the expandable balloon so as to at least partially deflate the expandable balloon.

11. The ablation system of claim 10, further comprising a metering device connected to the exhaust lumen, wherein the metering device is configured to control a rate at which the reaction product is released from the expandable balloon to maintain a pressure in the expandable balloon.

12. The ablation system of claim 10, further comprising a vacuum generator connected to the exhaust lumen, the vacuum generator being configured to apply a vacuum to the exhaust lumen so as to exhaust the reaction product from the expandable balloon.

13. The ablation system of claim 1, further comprising at least one of:
a temperature sensor configured to monitor a temperature of the exothermic reaction; and
a pressure sensor configured to monitor a pressure in the expandable balloon,
wherein, in response to the monitored temperature or the monitored pressure, the ablation system is configured to exhaust a reaction product of the first ablation reagent and the second ablation reagent from the expandable balloon so as to prevent at least one of thermal injury to bodily tissue or over-inflation of the expandable balloon.

14. A method comprising:
delivering a first ablation reagent into a catheter inserted into at least a portion of a human airway, the catheter extending from a proximal end to a distal end and including an expandable balloon adjacent the distal end;
delivering a second ablation reagent into the catheter; and
generating an exothermic reaction by mixing the first and second ablation reagents to heat the expandable balloon.

15. The method of claim 14, wherein the expandable balloon is inserted into at least a portion of a bronchial tree, and delivering the first ablation reagent and delivering the second ablation reagent comprises delivering at least one of the first ablation reagent and the second reagent into the expandable balloon so as to expand the expandable balloon to conform to the portion of the bronchial tree into which the expandable balloon is inserted.

16. The method of claim 15, wherein the expandable balloon is inserted into at least one of a primary bronchi, a secondary bronchi, a tertiary bronchi, and a terminal bronchiole.

17. The method of claim 15, wherein delivering at least one of the first ablation reagent and the second reagent into the expandable balloon so as to expand the expandable balloon comprises delivering at least one of the first ablation reagent and the second reagent into the expandable balloon so as to expand the expandable balloon at least expanding until the expandable balloon contacts a wall of the bronchial tree about substantially an entire perimeter of the expandable balloon.

18. The method of claim 14, wherein delivering the first ablation reagent and delivering the second ablation reagent comprises combining the first ablation reagent and second ablation reagent at least one of in the expandable balloon or proximally of the expandable balloon so as to generate the exothermic reaction.

19. The method of claim 18, wherein combining the first ablation reagent and second ablation reagent so as to generate the exothermic reaction comprises combining the first ablation reagent and second ablation reagent so as to cause an exterior surface of the expandable balloon to increase to a temperature ranging from approximately 45 degrees Celsius to approximately 75 degrees Celsius for a period of at least 5 seconds.

20. The method of claim 14, wherein the first ablation reagent is selected from the group consisting of HCl, AcOH, and citric acid, and the second ablation reagent is an alkali metal hydroxide.

21. The method of claim 14, wherein delivering the first ablation reagent into the catheter comprises delivering the first ablation reagent into a first lumen of the catheter, and delivering the second ablation reagent into the catheter comprises delivering the second ablation reagent into a second lumen of the catheter that is different than the first lumen.

22. The method of claim 14, further comprising applying a vacuum to the catheter so as to exhaust a reaction product of the first ablation reagent and the second ablation reagent from the expandable balloon.

23. The method of claim 22, wherein applying the vacuum to the catheter comprises applying the vacuum to an exhaust lumen of the catheter that is different than a lumen configured to deliver the first ablation reagent and second ablation reagent to the expandable balloon.
